(19) United States
(12) Patent Application Publication
(10) Pub. No.: US 2020/0338090 A1
(43) Pub. Date: Oct. 29, 2020

(54) SEIZURE CONTROL COMPOSITIONS AND METHODS OF USING SAME

(71) Applicant: Marsh and Wang Medical Systems, LLC, Peoria, AZ (US)

(72) Inventor: Steven T. Marsh, Phoenix, AZ (US)

(73) Assignee: Marsh and Wang Medical Systems, LLC, Peoria, AZ (US)

(21) Appl. No.: 16/397,365

(22) Filed: Apr. 29, 2019

(51) Int. Cl. A61K 31/5513; A61K 31/08; A61K 31/515; A61P 25/08

(52) U.S. Cl. CPC A61K 31/5513; A61P 25/08; A61K 31/515; A61K 31/08

(57) ABSTRACT

An anti-seizure composition includes a therapeutically effective combination of a halogenated ether, a benzodiazepine, and a barbiturate. The halogenated ether may selected from one or more of isoflurane, desflurane and sevoflurane. The benzodiazepine may be diazepam and the barbiturate may be phenobarbital. Also, a method for treating a seizure event may include administering a therapeutically effective amount of the combination.

### TECHNICAL FIELD

The present invention relates to seizure control compositions and methods; more particularly, to compositions and methods for the treatment of status epilepticus; and still more particularly, to compositions and methods for the treatment of status epilepticus comprising a three-drug combination.

### BACKGROUND OF THE INVENTION

Terrorism, and other international conflicts, makes the use of chemical warfare agents against civilians and/or military personnel a concern that requires adequate preparation. Nerve agents used in chemical warfare often lead to organophosphate poisoning. Exposure to nerve agents such as G-Series agents (i.e tabun (GA), sarin (GB), cyclosarin (GF), soman (GD)) and V-Series agents (i.e VX gas) results in a cholinergic crisis. Specifically, the inhibition of acetylcholinesterase (AChE), which is the primary metabolic enzyme of the cholinergic neurotransmitter acetylcholine. The resulting buildup of acetylcholine leads to an excessive release of the excitatory neurotransmitter glutamate from neuronal cells. This abundance of glutamate causes hyperexcitability in the brain. One of the consequences of this hyperexcitability can be status epilepticus (SE). SE is a neurological emergency defined as either continuous seizure activity for greater than 30 minutes or recurrent seizure activity without a return to a baseline between events. SE is independently associated with high mortality and morbidity rates. SE needs to be treated immediately and effectively in order to prevent adverse outcomes including cognitive disorders, subsequent epilepsy and even death. The release of a chemical nerve agent in a civilian and/or military setting would result in nearby treatment facilities being overwhelmed with a significant number of victims presenting clinical signs of SE. While the above description is directed toward exposure to chemical agents during warfare, it should be noted that SE can occur without any known cause.

Exposure by any route is considered extremely neurotoxic. If an agent were inhaled, the estimated LCₜ50 ranges from 10 mg-min/m³ for VX to 40 mg-min/m³ for GA in any exposed population. If an agent were to come in direct contact with an individual's skin, one drop (40-50 μl) of VX can be fatal; while 1-10 mL of GA, GB, or GD can be fatal. The onset and severity of symptoms are dependent upon the concentration of the agent and route of exposure.

Current treatments in the field include military Mark I NAAK kits (Nerve Agent Antidote Kit) which contain autoinjectors with atropine and pralidoxime chloride (2-PAM). Atropine may treat seizures but only in a very narrow time window, i.e. within 5 minutes of exposure, which is mainly important for the prevention of systemic effects of the nerve agent (i.e. muscle contractions, excessive production of mucous, tears, saliva and sweat). 2-PAM, an oxime for disassociating the nerve agent from the cholinesterase molecule, does not have a theraputic effect on SE. Thus, the Mark I NAAK kits are ineffective is arresting and treating status epilepticus.

A separate autoinjector of the benzodiazepine, diazepam (DZP), is also available but studies of SE have shown that benzodiazepines alone will not be effective in up to 40% of cases. This 40% of cases are medically considered to be refractory status epilepticus (RSE).

RSE cases markedly complicate the logistics of acute treatment. Currently, there is no way to differentiate on presentation which cases will be responsive to benzodiazepine treatment versus those that will become refractory. Triaging could only be done after DZP is given and sufficient time is allowed to distinguish between the two populations. Current treatment protocols would then require placement of the RSE victims in barbiturate/anesthetic coma for hours/days to abate ictal activity. With mass nerve gas exposures, this would quickly saturate available intensive care unit resources to maintain such cases. Moreover, mortality, despite such RSE treatments, remains at 23%—although SE induced by nerve gas may result in higher rates. Additionally, survivors are more likely to have cognitive declines (85% of RSE versus 61% of responsive SE) and post-SE epilepsy (87.5% versus 22%).

Thus, what is needed is a more effective first-line SE treatment form that indiscriminately aborts responsive and refractory SE cases. This treatment would also have a favorable safety profile with minimal side effects, improve overall mortality rates, and minimize post-SE neurological deficits and de novo epilepsy cases.

### SUMMARY OF THE INVENTION

Briefly described, an anti-seizure composition comprises a therapeutically effective combination of a halogenated ether, a benzodiazepine, and a barbiturate. The halogenated ether may selected from one or more of isoflurane, desflurane and sevoflurane. The benzodiazepine may be diazepam and the barbiturate may be phenobarbital. In accordance with an aspect of the present invention, the halogenated ether may have a concentration between about 0.1% and about 5%.

In a further aspect of the present invention, a method for treating a seizure event may include administering a therapeutically effective amount of a combination of a halogenated ether, a benzodiazepine, and a barbiturate. The halogenated ether may selected from one or more of isoflurane, desflurane and sevoflurane. The benzodiazepine may be diazepam and the barbiturate may be phenobarbital. The halogenated ether has a concentration between about 1% and about 5%, and may be administered via one or more of inhalation, subcutaneous injection, oral ingestion, intravenous injection, intramuscular injection, intraperitoneal injection and transdermal absorption.

### BRIEF DESCRIPTION OF THE DRAWINGS

The above-mentioned and other features are advantages of this invention, and the manner of attaining them, will become apparent and be better understood by reference to the following description of the invention in conjunction with the accompanying drawings, wherein:

FIG. 1 shows EEG traces of two rats following induction of status epilepticus (SE);

FIG. 2 shows EEG traces for the same rats shown in FIG. 1, with rat 1 having been administered a three-component drug regimen in accordance with the present invention;

FIG. 3 shows EEG traces for the same rats shown in FIGS. 1 and 2, with both rats having been administered the three-component drug regimen; and

FIG. 4 shows EEG traces for the same rats shown in FIGS. 1-3, two days after induction of SE and administration of the three-component drug.

### DETAILED DESCRIPTION

As described above, typical treatment of SE includes administration of a combination of two standard antiepilepsy drugs (AEDs), diazepam (DZP) and phenobarbital (PB). Alternatively, such as in a clinical setting, DZP may be first administered to abort the ictal activity. If DZP alone is ineffective, PB may then be added. In either event, this combination controls the immediate onset of SE only about 60% of the time. The remaining roughly 40% of cases are referred to as refractory SE (RSE). In these instances, patients are at higher risk for lower cognitive function, recurrent unprovoked seizures (i.e. epilepsy), and death.

In accordance with an aspect of the present invention, a treatment for status epilepticus (SE) comprises a therapeutically effective combination of a halogenated ether, a benzodiazepine such as DZP, and a barbiturate such as PB. The halogenated ether is selected from one or more of isoflurane, desflurane and sevoflurane, and in accordance with one aspect, is isoflurane. The halogenated ether may be administered via any suitable delivery method, including but not limited to through inhalation, subcutaneous injection, oral ingestion, intravenous injection, intramuscular injection, intraperitoneal injection and transdermal absorption. The concentration of the halogenated ether may range from about 0.1% to about 5%, more particularly about 1.5% to about 3.5%, and still more particularly about 2% to about 3%. When the ether is inhaled, the remainder of the inhalation gas is typically oxygen.

As set forth in the below example, a treatment of status epilepticus comprises administration of a therapeutically effective amount of the three-drug combination. By way of example and without limitation thereto, one example of an effective three-drug combination includes DZP: PB: Isoflurane.

### Experimental Example 1

Adult male Sprague Dawley rats (approximately 200-250 grams) underwent surgery to have epidural screw electrodes implanted in the skull for EEG recording to detect electrographic changes induced by seizures. The rats were housed singly after surgery, with food and water available ad libitum, with a 24 hour diurnal light cycle maintained, with lights on from 0700 to 1900 each day. All animal procedures were conducted in accordance with National Institute of Health's (NIH) Guide for the Care and Use of Laboratory Animals: Eighth Edition (2011), The Association for Assessment and Accreditation of Laboratory Animal Care Guidelines and the Institutional Animal Care and Use Committee.

Treatment response and concentrations were based on a single animal model for SE. Rate and intensity of the development of chronic epilepsy was determined by chronically monitoring the rats for 3 months following the induction and treatment of SE. This time duration was chosen to detect progressive long term changes as the result of the treatments. An episode of SE was chemically induced and the rats were then treated with a systemic injection of the standard antiepileptic drugs DZP and PB in combination with inhaled isoflurane/oxygen to abort later stages of RSE, as measured by time and EEG characteristics.

A lithium-pilocarpine protocol was used to generate Generalized Convulsive Status Epilepticus (GCSE) in the rats. Tylenol (1-2 mg per ml) was added to drinking water the day before surgery and for three days post-operatively. One week after electrode implantation surgery, a baseline EEG was recorded for 15 minutes. Status epilepticus was then induced by an intraperitoneal (IP) injection of lithium chloride (3 mmol/kg) followed by subcutaneous (SC) injection of pilocarpine (30 mg/kg) 20-24 hours later. Following injection of pilocarpine, the EEG of each rat was monitored continuously by being placed in a recording cage and connected to a clinical EEG machine by a flexible cable suspended from the top of the cage. The cage was equipped with an interposed commutator system to allow the rats to turn freely without twisting the cable.

GCSE was stopped by an IP injection of a two-component drug combination of the standard antiepilepsy drugs (AEDs) diazepam (DZP) and phenobarbital (PB), along with inhalation of an isoflurane/oxygen mixture in accordance with the present invention. For example, and without limitation thereto, each rat was administered 10 mg/kg DZP, 25 mg/kg PB and 2-3% isoflurane concentration after each animal progressed to complete ictal pattern of SE on the EEG. Each animal was continuously monitored for the next 4 to 6 hours. Following the acute experiment, each rat was chronically monitored.

The rats were fed a mush containing rat biscuits and water and were continuously monitored with EEG recording for 12 weeks to detect the development of chronic epilepsy. The number, frequency, and duration of seizures was recorded for each rat every day.

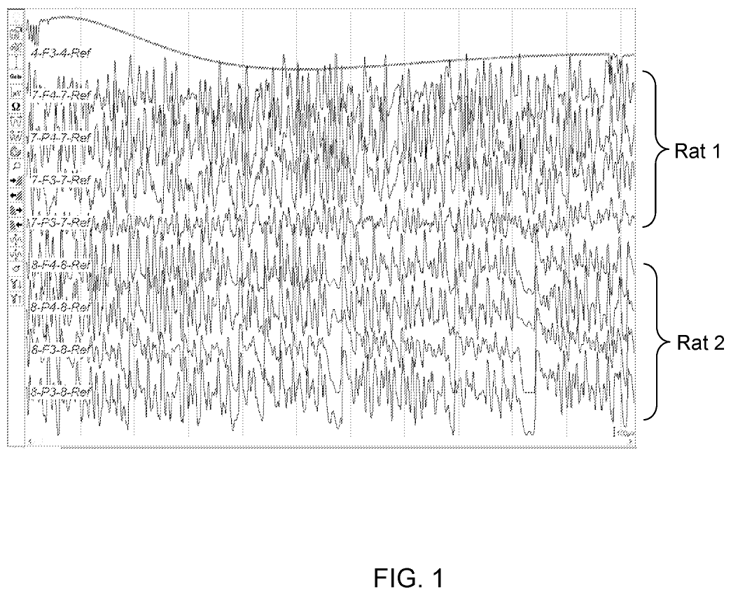

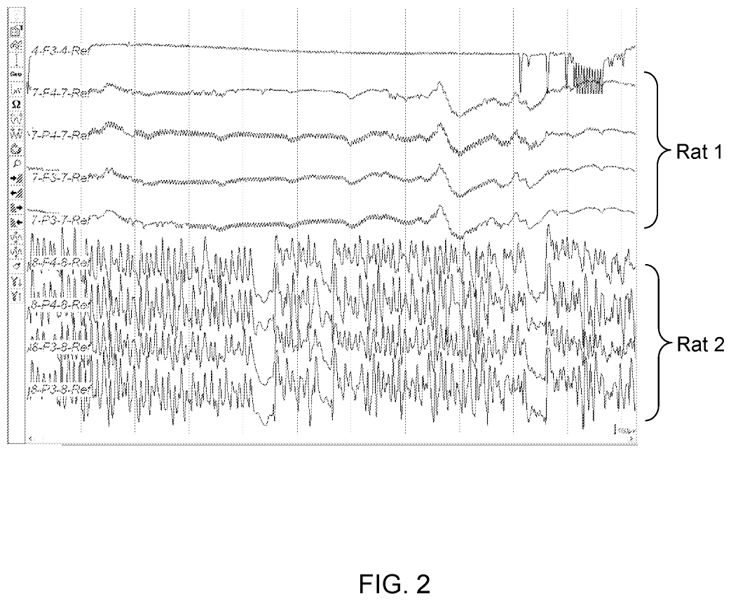

Turning now to the figures, FIG. 1 shows EEG traces of two rats following induction of status epilepticus (SE). Traces 1-4 are for rat 1, while traces 5-8 are for rat 2. As can be seen, each rat is in a completely ictal state of SE. FIG. 2 shows traces for the same rat 1 and rat 2, with rat 1 having been administered the three-component drug regimen in accordance with the present invention, consisting of 10 mg/kg DZP, 25 mg/kg PB and 2-3% isoflurane in oxygen. It should be understood by those skilled in the art that the above composition is exemplary and is not to be seen as limiting in any way. It should be further noted that alternative concentrations or ranges of concentrations may be utilized provided such compositions are therapeutically effective in accordance with the present invention.

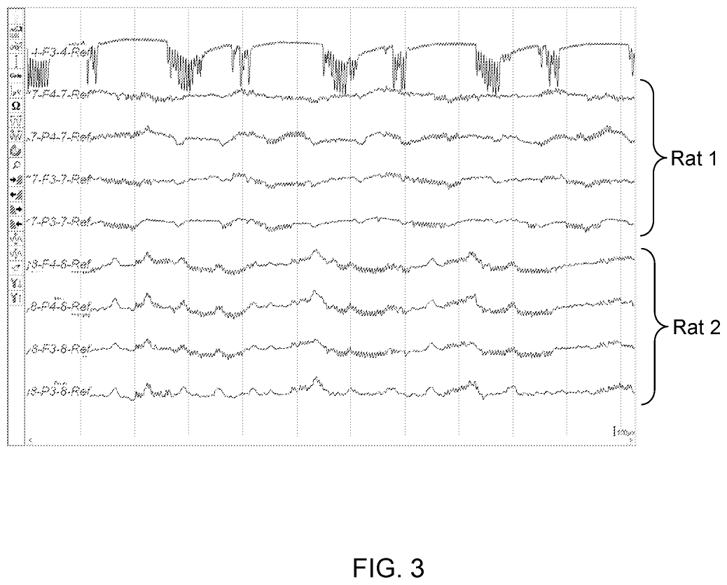

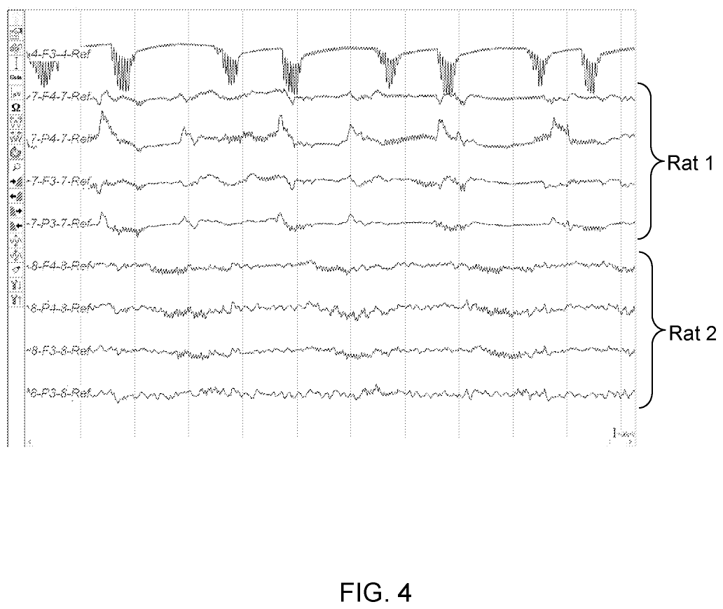

As can be seen, the EEG traces from rat 1 show a return to normal EEG patterns while rat 2 continues in the completely ictal state. FIG. 3 shows traces for the same rats 1 and 2, with each rat having been administered the three-component drug regimen in accordance with the present invention. As can be seen, each rat has returned to normal EEG background patterns. Finally, FIG. 4 shows EEG traces two days after induction of SE and administration of the three-component drug regimen. Each rat continues to exhibit normal EEG activity with no evidence of seizures over the course of approximately 48 hours. Rats 1 and 2 were followed for a further 17 weeks with no recurrence of spontaneous seizures or other indications of epilepsy.

In contrast, rats given only standard AED's administered 60 minutes following the pilocarpine injection do not affect the observed ictal EEG morphology. The animals continue uninterrupted in the ictal pattern until a point when the brain resources begin to become exhausted. It is then that flatline “breaks” in between the ictal pattern become wider and more frequent over the next few hours. The flatline breaks then become continuous, marked with an occasional sharp spike. These spikes are known clinically as PED's or periodic epileptiform discharges. The presence of PED's indicates that death is imminent and the rodent will be deceased a total of 4.5-5 hours following the pilocarpine injection, despite receiving AED's. While the above was described with reference to a lithium/pilocarpine induction model, it should be noted that similar ictal patterns may be caused by other models/chemical agents, with complete ictal activity progressing at different rates/times.

While the invention has been described by reference to various specific embodiments, it should be understood that numerous changes may be made within the spirit and scope of the inventive concepts described. Accordingly, it is intended that the invention not be limited to the described embodiments, but will have full scope defined by the language of the following claims.

1. An anti-seizure composition comprising a therapeutically effective combination of a halogenated ether, a benzodiazepine, and a barbiturate.

2. The anti-seizure composition in accordance with claim 1 wherein the halogenated ether is selected from one or more of isoflurane, desflurane and sevoflurane.

3. The anti-seizure in accordance with claim 2 wherein the halogenated ether is isoflurane.

4. The anti-seizure composition in accordance with claim 1 wherein the benzodiazepine is diazepam.

5. The anti-seizure composition in accordance with claim 1 wherein the barbiturate is phenobarbital.

6. The anti-seizure composition in accordance with claim 1 wherein the halogenated ether has a concentration between about 0.1% and about 5%.

7-13. (canceled)
